(19) United States
(12) Patent Application Publication
(10) Pub. No.: US 2025/0295641 A1
(43) Pub. Date: Sep. 25, 2025

(54) TREATMENT FOR ASD

(71) Applicant: STALICLA SA, Geneva (CH)

(72) Inventors: Lynn Durham, Geneva (CH); Laura Pérez-Cano, Barcelona (ES); Emre Guney, Barcelona (ES); Francesco Sirci, Barcelona (ES); Xavier Gallego, Barcelona (ES); Samuel Valentini, Barcelona (ES); Jose Manuel Hidalgo, Barcelona (ES)

(21) Appl. No.: 19/089,337

(22) Filed: Mar. 25, 2025

(30) Foreign Application Priority Data
Mar. 25, 2024 (CH) CH000317/2024

(51) Int. Cl. A61K 31/437; A61K 31/196

(52) U.S. Cl. CPC A61K 31/437; A61K 31/196

(57) ABSTRACT

The invention relates to a pharmaceutical composition comprising ibudilast and bumetanide for use in the treatment of autism spectrum disorder (ASD), wherein the composition is administered to a patient showing an overactivation of an NF-κB pathway.

### FIELD OF THE INVENTION

The invention relates to treatments of ASD subgroups.

### BACKGROUND

Autism spectrum disorder (ASD) is an etiologically and clinically heterogenous group of neurodevelopmental disorders (NDDs). According to the Diagnostic and Statistical Manual of Mental Disorders, Fifth Edition (DSM-5) criteria, the diagnosis of ASD is based on core behavioral symptoms such as impaired social communication and repetitive and restrictive behaviors. Based on recent estimates in the United States and Europe, one out of 36 to 89 children aged around 8 years receives a diagnosis of ASD. Patients diagnosed with ASD, like many other NDDs, show a variety of clinical manifestations and genomic alterations, hindering the development of treatments that are effective at the population level for individuals with ASD. Consequently, there is still a high unmet need for effective treatments of ASD.

Given the broad spectrum of molecular etiologies underlying patients diagnosed with ASD, it is not surprising that no clinically proven treatment addressing the core symptoms of ASD exists to date. The drug treatments currently approved by FDA for use in ASD—risperidone and aripiprazole—address certain behavioral features like irritability rather than the core symptoms. Existing clinical trials target patients diagnosed on the basis of behavioral assessments, often without qualitatively accounting for either the core symptoms or other non-behavioral comorbidities. Several compounds, such as memantine and sulforaphane, although showing a clear improvement in individual patients, fail to achieve a clear positive response across the whole population of patients. Given the clinical, genetic, and molecular heterogeneity observed in ASD, several recent studies have thus aimed at characterizing disease subtypes in ASD, albeit relying mainly on behavioral data.

Earlier studies on prepubertal individuals with ASD investigated how morphometric features extracted from structural magnetic resonance imaging (MRI) and distances between standardized facial landmarks describing facial morphology vary across individuals with distinct cognitive and language skills. Along these lines, a study by Libero and colleagues suggested the existence of a subgroup of patients with ASD characterized by an enlarged head circumference in early childhood. In another study, behavioral data from 47 preteen children diagnosed with ASD and 58 typically developing (TD) age-matched children were analyzed to characterize potential cognitive subtypes within the ASD population. The authors assessed the fulfillment of various tasks related to non-social information processing skills including spatial working memory, response inhibition, facial recognition and affect. Using the measures obtained from these tasks, they built a random forest model and hinted at the existence of subgroups of individuals among both ASD and TD groups with small but significant differences in the resting-state functional connectivity MRIs.

Other studies used electronic medical records of patients older than 15 years old to delineate potential systems-level clinical manifestations across patients with ASD beyond the neurobehavioral criteria from the DSM. They grouped individuals with ASD based on the co-occurrence of medical comorbidities using hierarchical clustering, identifying various patient subgroups that predominantly have seizures, gastrointestinal and auditory disorders, and psychiatric disorders such as episodic mood disorders, bipolar disorder, depression, anxiety, and conduct disorders. More recently, in addition to medical records, they have expanded their analysis using data from healthcare claims, familial whole-exome sequencing, and neurodevelopmental gene expression to characterize a mechanistically defined subgroup of patients with different clinical, genetic, and transcriptomic characteristics. The results from these studies suggest the existence of ASD subgroups with distinct clinical and etiologic differences driven by different genetic and environmental contributions.

Lately, various bioinformatic and machine learning (ML)-based approaches have been explored to address the challenges posed by ASD clinical variability and genetic heterogeneity. Among these, DEPI® (Databased Endophenotyping Patient Identification) is a systems biology, multi-omics, and ML-driven platform designed for the identification of biologically enriched subgroups of patients with NDDs and the potential corresponding tailored treatments. DEPI integrates information on NDD-risk factors including genetic variants, differentially expressed genes in large-scale case-control studies, and comorbidities observed across patients with neurodevelopmental disorders, and uses this information to identify pathway-level perturbations associated with clinical manifestations observed in patients with NDDs.

Such an approach could therefore be used to identify subgroups of ASD patients and provide targeted treatments for these patient groups based on the underlying genetic and molecular disturbances.

### Objective Technical Problem

The objective technical problem is therefore the provision of treatments for ASD patients that target specific subgroups of patients sharing a common etiology.

### SUMMARY OF THE INVENTION

In one aspect, the invention relates to a pharmaceutical composition comprising ibudilast and bumetanide for use in the treatment of autism spectrum disorder (ASD), wherein the composition is administered to a patient showing an overactivation of an NF-κB pathway.

In another aspect, the invention relates to a kit comprising a dosage form comprising ibudilast and a dosage form comprising bumetanide for use in the treatment of autism spectrum disorder (ASD), wherein the dosage forms are administered to a patient showing an overactivation of an NF-κB pathway.

In yet another aspect, the invention relates to a method of treatment ASD, wherein an effective amount of ibudilast and an effective amount of bumetanide is administered to an ASD patient showing an overactivation of an NF-κB pathway.

### BRIEF DESCRIPTION OF THE FIGURES

FIG. 1 shows the PCA of gene expression profiles from ASD-Phen1 (n=10) and ASD-non-Phen1 (n=10) patients using A) statistically significantly differentially expressed genes and B) top 250 up- and down-regulated genes (sorting by Log2 (FC)) in ASD-Phen1 compared to ASD-non-Phen1.

### DETAILED DESCRIPTION

In one aspect, the invention relates to a pharmaceutical composition comprising ibudilast and bumetanide for use in the treatment of autism spectrum disorder (ASD), wherein the composition is administered to a patient showing an overactivation of an NF-κB pathway.

In another aspect, the invention relates to a kit comprising a dosage form comprising ibudilast and a dosage form comprising bumetanide for use in the treatment of autism spectrum disorder (ASD), wherein the dosage forms are administered to a patient showing an overactivation of an NF-κB pathway.

In yet another aspect, the invention relates to a method of treatment ASD, wherein an effective amount of ibudilast and an effective amount of bumetanide are administered to an ASD patient showing an overactivation of an NF-κB pathway.

The pharmaceutical composition and kit according to the invention comprise ibudilast. Ibudilast is an anti-inflammatory and neuroprotective oral agent, metabolized mainly by the liver, having the following chemical structure of Formula I

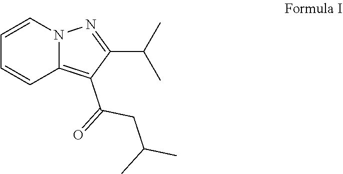 [SMILES: CC(C)CC(=O)c1c(C(C)C)nn2ccccc12]

Ibudilast is a phosphodiesterase (PDE) inhibitor, inhibiting mostly PDE4. The clinical efficacy of ibudilast has been proven for bronchial asthma and cerebrovascular disorders. Ibudilast is currently under clinical trial in the U.S. for progressive multiple sclerosis and other conditions such as amyotrophic lateral sclerosis and substances dependence (codes: AV-411 or MN-166).

The pharmaceutical composition or the dosage form of the kit according to the invention comprise between 2.5 mg and 50 mg ibudilast and are administered twice daily, so that the total daily dosage of ibudilast in the treatment according to the invention is between 5 and 100 mg ibudilast. These amounts are considered effective amounts. In preferred embodiments, the pharmaceutical composition or the dosage form of the kit comprise 5 or 10 mg ibudilast, so that ibudilast is preferably administered at a total daily dosage of 10 or 20 mg.

The pharmaceutical composition and kit according to the invention comprise bumetanide, also referred to as 3-(butylamino)-4-phenoxy-5-sulfamoylbenzoic acid. Bumetanide is an NKCC1 inhibitor and acts a loop diuretic. It is available under the tradenames bumex and burinex, among others. Its chemical structure is depicted below as Formuala II

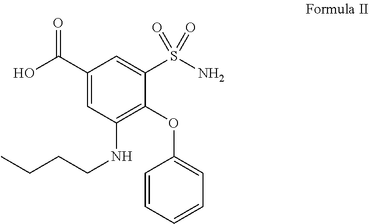 [SMILES: CCCCNc1cc(C(=O)O)cc(S(N)(=O)=O)c1Oc1ccccc1]

The pharmaceutical composition or the dosage form of the kit according to the invention comprise between 0.25 mg and 5 mg bumetanide and are administered twice daily, so that the total daily dosage of bumetanide in the treatment according to the invention is between 0.5 and 10 mg bumetanide. These amounts are considered effective amounts. In preferred embodiments, the pharmaceutical composition or the dosage form of the kit comprise 1 mg bumetanide, so that bumetanide is preferably administered at a total daily dosage of 2 mg.

In some embodiments, instead of bumetanide itself, the pharmaceutical composition or the dosage form of the kit according to the invention comprise derivatives of bumetanide, such as AqB007, AqB011, PF-2178, BUM13, BUM5 or bumepamine.

In a preferred embodiment, the treatment comprises administration of a total daily dosage of between 5 mg and 100 mg ibudilast and of a total daily dosage of between 0.5 and 10 mg bumetanide. In a particularly preferred embodiment, the treatment comprises administration of a total daily dosage of between 10 mg and 20 mg ibudilast and of a total daily dosage of 2 mg bumetanide.

The compositions and kits are for use in the treatment of ASD, wherein the treatment, i.e., the composition and dosage forms of the kit, is administered to a patient showing an overactivation of an NF-κB pathway.

Nuclear factor kappa-light-chain-enhancer of activated B cells (NF-κB) is a transcription factor involved in cellular responses to stimuli such as stress, cytokines, free radicals, heavy metals, irradiation or bacterial or viral antigens. NF-κB regulates expression of a large number of genes that are critical for the regulation of apoptosis, tumorigenesis, inflammation and various autoimmune disorders. NF-κB comprises a homo- or heterodimeric protein complex formed by the Rel-like domain-containing proteins RELA/p65, RELB, NFKB1 p105, NFKB1 p50 (the N-terminal processed product of the precursor p105), REL and NFKB2 p52, with the heterodimeric p65-p50 complex being the most abundant one. NF-κB activation occurs via two major signaling pathways: i) the canonical pathway; and ii) the non-canonical NF-κB signalling pathways. The canonical pathway mediates the activation of NF-κB1 p50, RELA and REL, and leads to rapid but transient NF-κB activation, whereas the non-canonical NF-κB pathway selectively activates p100-sequestered NF-κB members, predominantly NF-κB2 p52 and RELB, and is characteristically slow and persistent.

Without wanting to be bound by a theory, it is believed that consistent with its central role in inflammatory response NF-κB is involved in the etiology of the disorder in some ASD patients showing high levels of inflammation. Following inflammation, pro-inflammatory cytokines such as tumor necrosis factor (TNF) a, interleukin (IL)-13, and bacterial lipopolysaccharide (LPS) activate NF-κB, which leads to the transcription of genes involved in inflammation development and progression. Elevated serum levels of pro-inflammatory cytokines have been previously reported in some patients diagnosed with ASD. Overactivation of an NF-κB is therefore a useful marker to identify a subset of ASD patient characterized by high levels of inflammation and therefore susceptible to treatment with ibudilast.

Overactivation of an NF-κB pathway can be detected by any means known in the art. In one embodiment, the overactivation is detected by detecting an upregulation of the expression of at least twenty NF-κB-associated genes. The term “NF-κB-associated genes” is herein understood to refer to genes that are transcriptional target genes of NF-κB. The expression level NF-κB-associated genes can be measured by any means known in the art, e.g., RNA-seq, rt-PCR.

In one embodiment, the overactivation of an NF-κB pathway is determined by detecting the upregulation of at least 20 genes selected from the group comprising ABCA1, ABCB1, ABCB4, ABCB9, ABCC6, ABCG5, ABCG8, ADH1A, ADORA1, ADORA2A, AFP, AGER, AGT, AICDA, ALOX12, AMACR, AMH, ANGPT1, APOBEC2, APOC3, APOD, APOE, AQP4, AR, ARFRP1, ART1, ASPH, ASS1, ATP1A2, B2M, BACE1, BAX, BCL2, BCL2A1, BCL2L1, BCL2L11, BCL3, BDKRB1, BDNF, BLIMP1/PRDM1, BLNK, BLR1, BMI1, BMP2, BMP4, BNIP3, BRCA2, BTK, C3, C4A, C4BPA, C69, CALCB, CASP4, CCL1, CCL15, CCL17, CCL19. CCL2, CCL20, CCL22, CCL23, CCL28, CCL3, CCL4, CCL5, CCND1, CCND2, CCR5, CCR7, CD209, CD274, CD38, CD3G, CD40, CD40LG, CD44, CD48, CD54, CD80, CD83, CD86, CDK6, CDX1, CEBPD, CFB, CFLAR, CGM3, CHI3L1, CIDEA, COL1A2, CR2, CREB3, CRP, CSF1, CSF2, CSF3, CTSB, CXCL1, CXCL10, CXCL3, CXCL5, CXCL9, CYP19A1, CYP27B1, CYP2C11, CYP2E1, CYP7B1, DEFB2, DIO2, DMP1, DNASE1L2, E2F3, EBI3, EDN1, EGFR, ELF3, ENG, EPHA1, EPO, ERBB2, ERVWE1, F3, F8, FABP6, FAM148A, FAS, FASLG, FCER2, FCGRT, FGF8, FN1, FSTL3, FTH1, G6PC, GADD45B, GATA3, GBP1, GCLC, GCLM, GNAI2, GNB2L1, GNRH2, GRM2, GRO-beta, GRO-gamma, GSTP1, GZMB, HAMP, HAS1, HBE1, HBZ, HIF1A, HLA-B, HLA-G, HMGN1, HMOX1, HOXA9, HSD11B2, HSP90AA1, IER3, IFNB1, IFNG, IGFBP2, IGHE, IGHG1, IGHG2, IGHG4, IGKC, ligp1, IL10, IL11, IL12A, IL12B, IL13, IL17, IL1A, IL1B, IL1RN, IL2, IL23A, IL27, IL2RA, IL6, IL8, IL8RA, IL8RB, IL9, INHBA, IRF1, IRF2, IRF4, IRF7, JMJD3, JUNB, KC, KCNK5, KCNN2, KISS1, KITLG, KLK3, KLRA1, KRT15, KRT3, KRT5, KRT6B, LAMB2, LBP, LCN2, LEF1, LGALS3, LIPG, LTA, LTB, LYZ, MADCAM1, MBP, MDK, MMP1, MMP3, MMP9, MTHFR, MUC2, MYB, MYC, MYLK, MYOZ1, NCAM, NFKB1, NFKB2, NFKBIA, NFKBIE, NFKBIZ, NGFB, NK4, NLRP2, NOD2, NOS1, NOS2A, NOX1, NPY1R, NQO1, NR4A2, NRG1, NUAK2, OLR1, OPN1SW, OPRD1, OPRM1, ORM1, Osterix, OXTR, PAFAH2, PDGFB, PDYN, PENK, PGLYRP1, PGR, PI3KAP1, PIGF, plgR, PIK3CA, PIM1, PLA2, PLAU, PLCD1, PLK3, POMC, PPARGC1B, PRF1, PRKACA, PRKCD, PRL, PSMB9, PSME1, PSME2, PTAFR, PTEN, PTGDS, PTGS2, PTHLH, PTPN1, PTX3, PYCARD, RAG1, RAG2, RBBP4, REL, RELB, S100A4, S100A6, SAA1, SAA2, SAA3, SAT1, SCNN1A, SDC4, SELE, SELP, SELS, SENP2, SERPINA1, SERPINA2, SERPINA3, SERPINB1, SERPINE1, PAI-1, SERPINE2, SH3BGRL, SKALP, PI3, SKP2, SLC11A2, SLC16A1, SLC3A2, SLC6A6, Slfn2, SNAI1, SOD1, SOD2, SOX9, SPI1, SPP1, ST6GAL1, ST8SIA1, STAT5A, TACR1, TAP1, TAPBP, TCRB, TERT, TFEC, TFF3, TGM1, TGM2, TICAM1, TLR2, TLR9, TNC, TNF, TNFAIP3, TNFRSF4, TNFRSF9, TNFSF10, TNFSF13B, TNFSF15, TNIP1, TNIP3, TP53, TRAF1, TRAF2, TREM1, TRPC1, TWIST1, UPK1B, UPP1, VCAM1, VEGFC, VIM, WT1, XIAP and YY1.

In a preferred embodiment, the overactivation of an NF-κB pathway is determined by detecting the upregulation of at least one gene selected from the group consisting of B2M, BCL2A1, BRCA2, C3, CD48, CFB, F8, FAS, GADD45B, IL1B, IL1RN, KRT5, LGALS3, LYZ, NFKBIZ, NRG1, PSMB9, PTEN, S100A6, SAA2, SAT1, SERPINB1, SH3BGRL and TNIP3. In particularly preferred embodiment, the overactivation of an NF-κB pathway is detected by detecting the upregulation of at least six genes selected from the group consisting of B2M, BCL2A1, BRCA2, C3, CD48, CFB, F8, FAS, GADD45B, IL1B, IL1RN, KRT5, LGALS3, LYZ, NFKBIZ, NRG1, PSMB9, PTEN, S100A6, SAA2, SAT1, SERPINB1, SH3BGRL and TNIP3.

The skilled person knows how to determine the expression level of a gene, including an upregulation of the expression of the gene. An upregulation of the expression of a gene is herein detected if the expression level of a gene is higher than at least one standard deviation from the mean expression in a control population.

In another embodiment, the overactivation of the NF-κB pathway is determined by detecting an increased level of NF-κB protein in a sample of the patient. In a preferred embodiment, the level of nuclear NF-κB protein is detected since only nuclear NF-κB will contribute to promotion of the expression of associated genes.

In a preferred embodiment, the level of NF-κB protein is measured by an immunoassay, preferably by an enzyme-linked immunosorbent assay (ELISA). In one embodiment, the kit may be the TransAM NF-κB Family Kit (Catalog No. 43296, Active Motif, Inc.).

An overactivation of an NF-κB pathway is detected when the level of NF-κB protein measured is at least one standard deviation higher than the mean protein level in a control population.

In a preferred embodiment, the patient shows an overactivation of an NF-κB pathway and an overactivation of an NRF2 pathway.

Nuclear factor erythroid 2-related factor 2 (NRF2), also known as nuclear factor erythroid-derived 2-like 2, encoded by the NFE2L2 gene, is a master transcription factor regulator of antioxidative responses triggered, among other, by injury and inflammation. Several inter-related cellular pathways share NRF2 as a central core node of convergence, including the mammalian Target of Rapamycin (mTOR) pathway and the Phosphoinositide 3-kinase-Protein kinase B (PI3KAkt) pathway, both extensively reported to be disrupted in some patients with Autism Spectrum Disorder (ASD) and neurodevelopmental disorders (NDDs). Accordingly, dysregulation of the NRF2-related molecular pathways has been previously reported in some patients with ASD which respond to treatment with ibudilast (EP 3 785 733).

Overactivation of an NRF2 pathway can be detected by any means known in the art. In one embodiment, the overactivation is detected by detecting an upregulation of the expression of at least 10 NRF2-associated genes. The term “NRF2-associated genes” is herein understood to refer to genes that are transcriptional target genes of NRF2. The expression level of NRF2-associated genes can be measured by any means known in the art, e.g., RNA-seq, rt-PCR,

In one embodiment, the overactivation of an NRF2 pathway is determined by detecting an overexpression of at least 10 NRF2-associated genes selected from group comprising ABCB6, ABCB9, ABCC5, ACCN1, ACO1, ACTR10, ADAMTS12, ADO, AFG3L1P, AIFM2, AKIRIN2, ALOX12P2, ALPI, AMN1, ANKRD11, ANKRD30BL, ANO4, ARID3A, ARRDC3, ATXN1, ATXN3L, AZIN1, AZIN1, BCL2L11, BEND6, BEND6, BMP10, BRD2, C21orf33, C6orf106, C9orf25, C9orf5, CAMK2D. CAND1, CASC3, CCDC64, CD226, CD27, CD83, CDK17, CDK6, CEBPA, CHST11, CLIP4, CLLU1OS, CLTC, CMPK1, COL24A1, CPEB2, CPEB3, CREBZF, CWC27, DAD1, DCUN1D4, DENND4C, DGCR6L, DNAJA2, DST, DSTNP2, DUSP2, DUSP5, EHMT1, EIF4G3, ELN, EPB41, ERC2, EXOC7, FAM157A, FAM76B, FASTKD2, FECH, FLNB, FSD1L, FTH1, FTL, GABBR2, GATS, GCLC, GCLM, GCNT3, GDF15, GPI, GPNMB, GRM8, GSR, GSTM5, GSTP1, HBB, HBE1, HERC1, HGD, HIF1A, HIST1H4H, HMOX1, HMOX1, HRASLS2, HTATIP2, HTRA3, IFRD1, IFT74, IGF2R, IPO7, IRF2, IRF2BPL, KCNN3, KEAP1, KIAA1522, KIFC3, LBR, LINC00273, LINC00299, LOC100130451, LOC100132891, LOC100507557, LOC147646, LOC284661, LOC284801, LOC338758, LOC338799, LOC440461, LOC643723, LOC646329, LRP8, LRRC8D, LY9, MAFG, MAFG, MAPRE3, MAPT, ME1, MESDC1, MFSD11, MIAT, MIR365A, MIR617, MKLN1, MOV10L1, MPPE1, MSL3, MTF2, MYC, NES, NEUROD4, NFE2L2, NKAIN1, NPLOC4, NQO1, NUMBL, NUP153, OR2AT4, P2RY10, PARN, PDCD1LG2, PDCD6IP, PEX5L, PGRMC2. PIP5K1C, PIR, PLA2G6, PMAIP1, PMAIP1, PMF1, PPARGC1B, PPIF, PRDM1, PRDX1, PRKACB, PRKCB, PSMA3, PTGES3, PVRL1, PVT1, RAB10, RAB35, RASAL3, RASSF6, RCAN1, RFFL, RNF213, RNF220, ROCK1, RSPH6A, RXRA, SAR1B, SEC61B, SEMA7A, SEMA7A, SETBP1, SH2D6, SLAMF7, SLC14A2, SLC25A25, SLC3A2, SLC48A1, SLC7A11, SLC9A7P1, SLCO5A1, SORBS2, SPRY3, SQSTM1, SQSTM1, SQSTM1, SRXN1, SSH1, ST6GALNAC1, STARD13, STXBP4, SUMO1P1, TANK, TBL1X, TBXAS1, TCL6, TEC, TEC, TFE3, THBS1, TKT, TMEM121, TMTC3, TNFRSF1A, TNFRSF8, TNFSF14, TRIM56, TSC22D1, TXN, TXNRD1, TXNRD1, UBC, UBE2E2, UNKL, VCP, VEZF1, VTRNA1-1, WDR81, WIPI2, YWHAG, ZFAT, ZMYND8, ZNF148, ZNF3, ZNF469 and ZNF673.

In a preferred embodiment, the overactivation of an NRF2 pathway is detected by detecting the upregulation of at least one gene selected from the group consisting of ACTR10, AZIN1, CPEB3, FECH, HBB, MSL3, NFE2L2, PMAIP1, PSMA3, PTGES3, SLC9A7P1 and TANK.

In another embodiment, the overactivation of the NRF2 pathway is determined by detecting an increased level of NRF2 protein in a sample of the patient. In a preferred embodiment, the level of nuclear NRF2 protein is detected since only nuclear NRF2 will contribute to promotion of the expression of associated genes.

In a preferred embodiment, the level of NRF2 protein is measured by an immunoassay, preferably by an enzyme-linked immunosorbent assay (ELISA). In one embodiment, the kit may be the Human Nuclear factor erythroid 2-related factor 2 (NFE2L2) ELISA kit (Catalog No. CSB-EL015752HU, Cusabio, TX) or the Human NRF2 ELISA Kit (Catalog No. EH348RB, Invitrogen, CA).

An overactivation of the NRF2 pathway is detected when the level of NRF2 protein measured is at least one standard deviation above the mean protein level in a control population.

According to the invention, a gene expression level or a protein level can be determined in any suitable sample. In preferred embodiments, the sample is a blood sample, a plasma sample, a peripheral blood mononuclear cell sample, a saliva sample or a urine sample. The sample may be processed or purified prior to use according to the invention. In a preferred embodiment, the sample is a peripheral blood mononuclear cell sample.

### EXAMPLES

### Example 1: Differential Gene Expression Analysis

RNA-seq analysis was conducted from blood samples obtained from 20 patients with ASD including: i) 10 patients classified as ASD phenotype 1 (ASD-Phen1) (positive for two primary criteria: enlarged head circumference (above the 75th percentile) within the first two years of life, and systematic aggravation of ASD behavioral symptoms during episodes of immune challenges such as fever and infection events (e.g., acute inflammation); and therefore expected to respond to a pharmaceutical composition comprising ibudilast and bumetanide; and ii) 10 age-matched participants not classified as ASD-Phen1 (a.k.a. ASD-non-Phen1), thus not expected to respond to such pharmaceutical composition. From each participant, two tubes each with 2.5 ml of whole blood in PAXGene® RNA tubes were delivered and sequenced at Omega Bioservices facilities in Georgia (US). The RNA extraction was performed using the QIAGEN PAXgene Blood RNA Kit/Mag-Bind® PX Blood RNA 96 Kit and the ERCC Ex-fold RNA reagent (Cat: 4456739) was added to each sample. Depletion of rRNA and hgbRNA and the RNA-seq library preparation was performed using the Illumina TruSeq Stranded Total RNA with Ribo-Zero Globin kit. The samples were then sequenced using the Illumina NovaSeq 6000 sequencer, with a 2×150 bp configuration.

The two blood samples obtained from each patient were sequenced in two different batches and considered as technical replicas. Quality control procedures were applied to confirm the absence of batch effects among the samples, by performing Principal Component Analyses (PCAs) using prcomp R function (stats package).

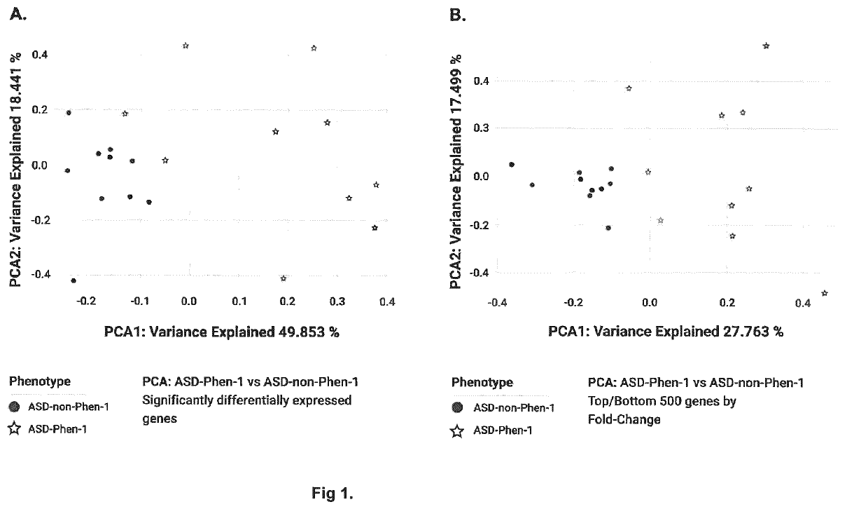

A differential gene expression analysis between ASD-Phen1 versus ASD-non-Phen1 was then conducted in order to characterize the ASD-Phen1-specific disease transcriptomic signature. R DESeq2 package was used for the differential gene expression analysis, using the option CollapseReplicates following recommended setup for multiple sequencing runs from the same extracted RNA amount, to increase statistical strength without introducing confounding batch effects. After the differential expression analysis, two PCA plots were produced to assess the power of differentially expressed genes to discriminate ASD-Phen1 vs. ASD-non-Phen1 individuals, first using differentially expressed genes with an adjusted p-value (according to the Benjamini and Hochberg correction method) under 0.05, and secondly using the top 250 up-/down-regulated genes (sorted by Log2 (FC)); this was done by considering only genes with median expression level across samples greater than 10 reads (FIG. 1). The first two PCA coordinates were subsequentially used as features to train a logistic regression model to classify the two phenotypes using their transcriptomic profile. The models were validated using a leave-one-out strategy where at each iteration a sample is classified using the others as a training set. The performances are computed as the number of correct classified samples in the whole dataset. To validate the procedure, we implemented a permutation test over 1,000 iterations where at each iteration phenotypes were randomly shuffled, and the classification score was computed to obtain their null distribution. This permutation testing using a leave-one-out cross-validation confirmed the significance of the observed stratification as compared to the null distribution (p=0.01).

### Example 2: Gene Set Enrichment Analyses

EnrichR, a comprehensive gene set enrichment analysis web server, was used to explore the pathway enrichment of differentially expressed genes (adjusted p-value below 0.05) in ASD-Phen1 vs. ASD-non-Phen1. Gene sets and pathways in MsigDB Hallmark 2020, the KEGG 2021 Human, the Reactome 2022, and the WikiPathway 2021 Human databases were used in this analysis, which showed statistically significant enrichment (adjusted p-value <0.05) of differentially expressed genes on pathways related to the activation of NF-κB and downstream pro-inflammatory cascades (Table 1), including the NF-κB-associated gene BCL2A1 (log 2FC=1.41; p-value=2.2E-05; adjusted p-value=0.043).

TABLE 1
NF-κB pathways significantly enriched for genes
showing a statistically significant differential
expression in ASD-Phen1 vs. ASD-non-Phen1

| | | | Adjusted | Odds |
|---|---|---|---|---|
| GeneSet_DB | Term | p-value | p-value | Ratio |
| MsigDB_{—} | TNF-alpha | 0.004 | 0.017 | 24.99 |
| Hallmark_2020 | Signaling via | | | |
| | NF-κB | | | |
| Reactome_{—} | MyD88:MAL(TIRAP) | 0.001 | 0.026 | 45.18 |
| 2022 | Cascade | | | |
| | Initiated On | | | |
| | Plasma | | | |
| | Membrane R- | | | |
| | HSA-166058 | | | |
| Reactome_{—} | Toll Like | 0.002 | 0.026 | 35.96 |
| 2022 | Receptor 4 | | | |
| | (TLR4) | | | |
| | Cascade R- | | | |
| | HSA-166016 | | | |
| Reactome_{—} | Toll-like | 0.003 | 0.026 | 30.98 |
| 2022 | Receptor | | | |
| | Cascades R- | | | |
| | HSA-168898 | | | |
| WikiPathway_{—} | Photodynamic | 0.017 | 0.028 | 65.22 |
| 2021_Human | Therapy- | | | |
| | Induced NF-κB | | | |
| | Survival | | | |
| | Signaling | | | |
| | WP3617 | | | |
| Reactome_{—} | MyD88 | 0.008 | 0.045 | 138.71 |
| 2022 | Deficiency | | | |
| | (TLR2/4) R- | | | |
| | HSA-5602498 | | | |
| Reactome_{—} | Regulation Of | 0.009 | 0.045 | 130.54 |
| 2022 | TLR By | | | |
| | Endogenous | | | |
| | Ligand R-HSA- | | | |
| | 5686938 | | | |
| Reactome_{—} | IRAK4 | 0.009 | 0.045 | 130.54 |
| 2022 | Deficiency | | | |
| | (TLR2/4) R- | | | |
| | HSA-5603041 | | | |
| Reactome_{—} | TRAF6- | 0.012 | 0.050 | 92.44 |
| 2022 | Mediated NF- | | | |
| | κB Activation | | | |
| | R-HSA- | | | |
| | 933542 | | | |

Gene set enrichment analysis (GSEA) was also performed for the ASD-Phen1 vs. ASD-non-Phen1 differential expressed genes with the aim to validate the consistency with NF-κB related pathways enrichment among ASD-Phen1. The fgsea R package v1.10.1 was used to run the analysis. A total of 15,497 gene ontology (GO) and canonical pathways (CP) were considered in the analysis as provided by the human MSigDB version 7.0 from the (https://data.broadinstitute.org/gsea-msigdb/msigdb/release/7.0/). A Fisher's exact test was used to confirm a significance over-representation of NF-κB-related pathways (i.e., those implicating NF-κB) among pathways enriched (Benjamini-Hochberg adjusted p-value <0.05) for genes differentially expressed in ASD-Phen1 patients (p-value=7E-15; 95% conf. inter. =2,41-4,17; odd ratio=3.19).

The association between the ASD-Phen1 specific transcriptomic disease signature and an over-activation of the NF-κB and NRF2 transcription factors was then evaluated by assessing the enrichment (based on the two-sided Kolmogorov-Smirnov test) of NF-κB and NRF2 high-confidence targets among top 250 genes with greatest up-or down-regulation (i.e., Log2 (FC)) when comparing gene expression between ASD-Phen1 and ASD-non-Phen1 patients. The ks.test R function (stats package) was used to calculate the enrichment score(ES) and the associated significance (two-tailed test). Positive or negative ES values indicate that NF-κB/NRF2 gene targets tend to be up-or down-regulated in the transcriptomic signature. Both NF-κB and NRF2 target genes were found to be significantly enriched towards up-regulated genes in the ASD-Phen1 group (NF-κB: ES=0.24 and p-value=<1E-9; NRF2: ES=0.21 and p-value=5.6E-9).

A total of twenty-four NF-κB transcriptional target genes, in particular B2M, BCL2A1, BRCA2, C3, CD48, CFB, F8, FAS, GADD45B, IL1B, IL1RN, KRT5, LGALS3, LYZ, NFKBIZ, NRG1, PSMB9, PTEN, S100A6, SAA2, SAT1, SERPINB1, SH3BGRL and TNIP3, were found to be differentially up-regulated genes in ASD-Phen1 (p-value <0.05). By performing some power calculations, we estimated that only twenty NF-κB transcriptional target genes would have been needed to be found to be differentially up-regulated in ASD-Phen1 (p-value <0.05) to reach statistical significance in the enrichment analysis (p-value <0.05). In addition, a total of six genes, in particular BCL2A1, TNIP3, NRG1, C3, IL1RN and KRT5, were found to be within the top 250 most up-regulated genes in ASD-Phen1 patients.

A total of twelve NRF2 transcriptional target genes, in particular ACTR10, AZIN1, CPEB3, FECH, HBB, MSL3, NFE2L2, PMAIP1, PSMA3, PTGES3, SLC9A7P1 and TANK, were found to be differentially up-regulated genes in ASD-Phen1 (p-value <0.05). By performing some power calculations, we estimated that only ten NRF2 transcriptional target genes would have been needed to be found to be differentially up-regulated in ASD-Phen1 (p-value <0.05) to reach statistical significance in the enrichment analysis (p-value <0.05). in addition, the HBB gene was found to be within the top 250 most up-regulated genes in ASD-Phen1 patients.

### Example 3: STP1 as a Suitable Drug Candidate to Revert NF-κB and NRF2 Over-Activation in ASD-Phen1

STP1 was obtained by equally diluting and mixing bumetanide and ibudilast to obtain a final 1:1 ratio of the two individual compounds in the DMSO solution. Commercial NPC (ATCC ACS-5004) and MCF-7 (ATCC HTB-22) cell lines were purchased from the ATCC LGC Standards® provider. The transcriptional signature of STP1 was then obtained by treating commercial NPC, and MCF-7 cell lines with a 5-u M final concentration of STP1 or control vehicle (DMSO) during 6 and 24 hours. In addition, LCLs derived from two ASD-Phen1 patients were treated with 5 u M of STP1 or DMSO for 48 hours. Each cell line was cultured following the provider's guidelines until a sufficient number of cells is reached to be seeded for treatment with STP1 or DMSO. All the treatments were performed in triplicates, seeding 50,000 cells/well at the two-time points. RNA was extracted from every single well obtaining a total of 36 samples for library preparation and sequencing. Total RNA was quantified using the Qubit fluorometric assay (Thermo Fisher Scientific). Libraries were prepared from 125 ng of total RNA and sequenced on a NovaSeq 6000 sequencer in single-end mode with a read length of 75 bp and 4M reads per sample. Bioinformatics analysis consisted of quality filtering, trimming, and alignment to the reference genome to generate raw data. The raw expression data were finally normalized and analyzed for each cell line. A final list of genes differentially expressed genes for each STP1 treatment vs. DSMO treatment was obtained across cell lines. A weighted-average merging method that considers the Spearman correlation across cell-type individual treatments was applied to generate drug-elicited transcriptomic responses for STP1 by combining individual treatments across cell lines. Finally, a total of three different transcriptional signatures were obtained: i) STP1 vs. DMSO treatment in MCF-7 and NPC; ii) STP1 vs. DMSO treatment in LCL derived from the first patient; iii) STP1 vs. DMSO treatment in LCL derived from the second patient.

The Kolmogorov-Smirnov test was then used to evaluate the effect of STP1 on the activity of NF-κB and NRF2 transcription factors, measured by the expression of their transcriptional targets in treated vs. untreated cell lines. Both NF-κB and NRF2 target genes were found to be significantly enriched towards down-regulated genes by STP1 in the three cases (Table 2).

TABLE 2
NRF2 and NF-κB gene targets enrichment analyses
results for different STP1 treated cell lines

| Transcriptomic | | | | |
|---|---|---|---|---|
| signatures | ES_{NRF2} | p-value | ES_{NF-κB} | p-value |
| commercial NPC | −0.13 | 8E−4 | −0.08 | 0.02 |
| and MCF7 cell lines | | | | |
| treated with STP1 | | | | |
| (5 μM) | | | | |
| First patient-derived | −0.19 | 2E−7 | −0.15 | 7E−7 |
| LCL treated | | | | |
| with STP1 (5 μM) | | | | |
| Second patient- | −0.17 | 4E−6 | −0.15 | 6E−7 |
| derived LCL treated | | | | |
| with STP1 (5 μM) | | | | |

1. A pharmaceutical composition comprising ibudilast and bumetanide for use in the treatment of autism spectrum disorder (ASD), wherein the composition is administered to a patient showing an overactivation of an NF-κB pathway.

2. A kit comprising a dosage form comprising ibudilast and a dosage form comprising bumetanide for use in the treatment of autism spectrum disorder (ASD), wherein the dosage forms are administered to a patient showing an overactivation of an NF-κB pathway.

3. The composition for use according to claim 1 or the kit for use according claim 2, wherein the overactivation of an NF-κB pathway is determined by detecting the upregulation of at least 20NF-κB-associated genes selected from the group comprising ABCA1, ABCB1, ABCB4, ABCB9, ABCC6, ABCG5, ABCG8, ADH1A, ADORA1, ADORA2A, AFP, AGER, AGT, AICDA, ALOX12, AMACR, AMH, ANGPT1, APOBEC2, APOC3, APOD, APOE, AQP4, AR, ARFRP1, ART1, ASPH, ASS1, ATP1A2, B2M, BACE1, BAX, BCL2, BCL2A1, BCL2L1, BCL2L11, BCL3, BDKRB1, BDNF, BLIMP1/PRDM1, BLNK, BLR1, BMI1, BMP2, BMP4, BNIP3, BRCA2, BTK, C3, C4A, C4BPA, C69, CALCB, CASP4, CCL1, CCL15, CCL17, CCL19, CCL2, CCL20, CCL22, CCL23, CCL28, CCL3, CCL4, CCL5, CCND1, CCND2, CCR5, CCR7, CD209, CD274, CD38, CD3G, CD40, CD40LG, CD44, CD48, CD54, CD80, CD83, CD86, CDK6, CDX1, CEBPD, CFB, CFLAR, CGM3, CHI3L1, CIDEA, COL1A2, CR2, CREB3, CRP, CSF1, CSF2, CSF3, CTSB, CXCL1, CXCL10, CXCL3, CXCL5, CXCL9, CYP19A1, CYP27B1, CYP2C11, CYP2E1, CYP7B1, DEFB2, DIO2, DMP1, DNASE1L2, E2F3, EBI3, EDN1, EGFR, ELF3, ENG, EPHA1, EPO, ERBB2, ERVWE1, F3, F8, FABP6, FAM148A, FAS, FASLG, FCER2, FCGRT, FGF8, FN1, FSTL3, FTH1, G6PC, GADD45B, GATA3, GBP1, GCLC, GCLM, GNAI2, GNB2L1, GNRH2, GRM2, GRO-beta, GRO-gamma, GSTP1, GZMB, HAMP, HAS1, HBE1, HBZ, HIF1A, HLA-B, HLA-G, HMGN1, HMOX1, HOXA9, HSD11B2, HSP90AA1, IER3, IFNB1, IFNG, IGFBP2, IGHE, IGHG1, IGHG2, IGHG4, IGKC, ligp1, IL10, IL11, IL12A, IL12B, IL13, IL17, IL1A, IL1B, IL1RN, IL2, IL23A, IL27, IL2RA, IL6, IL8, IL8RA, IL8RB, IL9, INHBA, IRF1, IRF2, IRF4, IRF7, JMJD3, JUNB, KC, KCNK5, KCNN2, KISS1, KITLG, KLK3, KLRA1, KRT15, KRT3, KRT5, KRT6B, LAMB2, LBP, LCN2, LEF1, LGALS3, LIPG, LTA, LTB, LYZ, MADCAM1, MBP, MDK, MMP1, MMP3, MMP9, MTHFR, MUC2, MYB, MYC, MYLK, MYOZ1, NCAM, NFKB1, NFKB2, NFKBIA, NFKBIE, NFKBIZ, NGFB, NK4, NLRP2, NOD2, NOS1, NOS2A, NOX1, NPY1R, NQO1, NR4A2, NRG1, NUAK2, OLR1, OPN1SW, OPRD1, OPRM1, ORM1, Osterix, OXTR, PAFAH2, PDGFB, PDYN, PENK, PGLYRP1, PGR, PI3KAP1, PIGF, plgR, PIK3CA, PIM1, PLA2, PLAU, PLCD1, PLK3, POMC, PPARGC1B, PRF1, PRKACA, PRKCD, PRL, PSMB9, PSME1, PSME2, PTAFR, PTEN, PTGDS, PTGS2, PTHLH, PTPN1, PTX3, PYCARD, RAG1, RAG2, RBBP4, REL, RELB, S100A4, S100A6, SAA1, SAA2, SAA3, SAT1, SCNN1A, SDC4, SELE, SELP, SELS, SENP2, SERPINA1, SERPINA2, SERPINA3, SERPINB1, SERPINE1, PAI-1, SERPINE2, SH3BGRL, SKALP, PI3, SKP2, SLC11A2, SLC16A1, SLC3A2, SLC6A6, Slfn2, SNAI1, SOD1, SOD2, SOX9, SPI1, SPP1, ST6GAL1, ST8SIA1, STAT5A, TACR1, TAP1, TAPBP, TCRB, TERT, TFEC, TFF3, TGM1, TGM2, TICAM1, TLR2, TLR9, TNC, TNF, TNFAIP3, TNFRSF4, TNFRSF9, TNFSF10, TNFSF13B, TNFSF15, TNIP1, TNIP3, TP53, TRAF1, TRAF2, TREM1, TRPC1, TWIST1, UPK1B, UPP1, VCAM1, VEGFC, VIM, WT1, XIAP and YY1.

4. The composition according to claim 1 or kit for use according to claim 2, wherein the overactivation of an NF-κB pathway is determined by detecting the upregulation of at least one, preferably at least six NF-κB-associated genes selected from the group consisting of B2M, BCL2A1, BRCA2, C3, CD48, CFB, F8, FAS, GADD45B, IL1B, IL1RN, KRT5, LGALS3, LYZ, NFKBIZ, NRG1, PSMB9, PTEN, S100A6, SAA2, SAT1, SERPINB1, SH3BGRL and TNIP3.

5. The composition for use according claim 1 or the kit for use according to claim 2, wherein the overactivation of the NF-κB pathway is determined by detecting an increased level of NF-κB protein in a sample of the patient.

6. The composition or kit for use according to claim 5, wherein the level of nuclear NF-κB protein is measured.

7. The composition or kit for use according to claim 5 or 6, wherein the level of NF-κB protein is measured by an immunoassay, preferably by an ELISA.

8. The composition or kit for use according to any of claims 1 to 7, wherein the composition is administered to a patient showing an overactivation of an NF-κB pathway and an overactivation of an NRF2 pathway.

9. The composition or kit for use according claim 8, wherein the overactivation of an NRF2 pathway is determined by detecting an overexpression of at least 10 NRF2-associated genes selected from the group comprising ABCB6, ABCB9, ABCC5, ACCN1, ACO1, ACTR10, ADAMTS12, ADO, AFG3L1P, AIFM2, AKIRIN2, ALOX12P2, ALPI, AMN1, ANKRD11, ANKRD30BL, ANO4, ARID3A, ARRDC3, ATXN1, ATXN3L, AZIN1, AZIN1, BCL2L11, BEND6, BEND6, BMP10, BRD2, C21orf33, C6orf106, C9orf25, C9orf5, CAMK2D, CAND1, CASC3, CCDC64, CD226, CD27, CD83, CDK17, CDK6, CEBPA, CHST11, CLIP4, CLLU1OS, CLTC, CMPK1, COL24A1, CPEB2, CPEB3, CREBZF, CWC27, DAD1, DCUN1D4, DENND4C, DGCR6L, DNAJA2, DST, DSTNP2, DUSP2, DUSP5, EHMT1, EIF4G3, ELN, EPB41, ERC2, EXOC7, FAM157A, FAM76B, FASTKD2, FECH, FLNB, FSD1L, FTH1, FTL, GABBR2, GATS, GCLC, GCLM, GCNT3, GDF15, GPI, GPNMB, GRM8, GSR, GSTM5, GSTP1, HBB, HBE1, HERC1, HGD, HIF1A, HIST1H4H, HMOX1, HMOX1, HRASLS2, HTATIP2, HTRA3, IFRD1, IFT74, IGF2R, IPO7, IRF2, IRF2BPL, KCNN3, KEAP1, KIAA1522, KIFC3, LBR, LINC00273, LINC00299, LOC100130451, LOC100132891, LOC100507557, LOC147646, LOC284661, LOC284801, LOC338758, LOC338799, LOC440461, LOC643723, LOC646329, LRP8, LRRC8D, LY9, MAFG, MAFG, MAPRE3, MAPT, ME1, MESDC1, MFSD11, MIAT, MIR365A, MIR617, MKLN1, MOV10L1, MPPE1, MSL3, MTF2, MYC, NES, NEUROD4, NFE2L2, NKAIN1, NPLOC4, NQO1, NUMBL, NUP153, OR2AT4, P2RY10, PARN, PDCD1LG2, PDCD6IP, PEX5L, PGRMC2, PIP5K1C, PIR, PLA2G6, PMAIP1, PMAIP1, PMF1, PPARGC1B, PPIF, PRDM1, PRDX1, PRKACB, PRKCB, PSMA3, PTGES3, PVRL1, PVT1, RAB10, RAB35, RASAL3, RASSF6, RCAN1, RFFL, RNF213, RNF220, ROCK1, RSPH6A, RXRA, SAR1B, SEC61B, SEMA7A, SEMA7A, SETBP1, SH2D6, SLAMF7, SLC14A2, SLC25A25, SLC3A2, SLC48A1, SLC7A11, SLC9A7P1, SLCO5A1, SORBS2, SPRY3, SQSTM1, SQSTM1, SQSTM1, SRXN1, SSH1, ST6GALNAC1, STARD13, STXBP4, SUMO1P1, TANK, TBL1X, TBXAS1, TCL6, TEC, TEC, TFE3, THBS1, TKT, TMEM121, TMTC3, TNFRSF1A, TNFRSF8, TNFSF14, TRIM56, TSC22D1, TXN, TXNRD1, TXNRD1, UBC, UBE2E2, UNKL, VCP, VEZF1, VTRNA1-1, WDR81, WIPI2, YWHAG, ZFAT, ZMYND8, ZNF148, ZNF3, ZNF469 and ZNF673.

10. The composition or kit for use according claim 8, wherein the overactivation of an NRF2 pathway is determined by detecting an overexpression of at least one NRF2-associated gene from the group consisting of ACTR10, AZIN1, CPEB3, FECH, HBB, MSL3, NFE2L2, PMAIP1, PSMA3, PTGES3, SLC9A7P1 and TANK.

11. The composition or kit for use according claim 8, wherein the overactivation of the NRF2-pathway is determined by detecting an increased level of NRF2 protein in a sample of the patient.

12. The composition or kit for use according to claim 11, wherein the level of nuclear NRF2 protein is measured.

13. The composition or kit for use according to claim 11 or 12, wherein the level of NRF2 protein is measured by an immunoassay, preferably by an ELISA.

14. The composition or kit for use according to any of claims 1 to 13, wherein the sample is a blood sample, a plasma sample, a peripheral blood mononuclear cell sample, a saliva sample or a urine sample.

15. The composition or kit for use according to any of claims 1 to 14, wherein the treatment comprises administration of a total daily dosage between 5 mg and 100 mg ibudilast and of a total daily dosage of between 0.5 and 10 mg bumetanide.
